(19) United States
(12) Patent Application Publication
(10) Pub. No.: US 2012/0237457 A1
(43) Pub. Date: Sep. 20, 2012

(54) ANTI-BACTERIAL ORAL CARE COMPOSITION AND METHOD FOR PRODUCING THE SAME

(76) Inventors: Shu-Cheng CHEN, Taipei City (TW); Ho-Yi Tsai, Taipei City (TW)

(21) Appl. No.: 13/052,077

(22) Filed: Mar. 20, 2011

(51) Int. Cl. A61K 8/73; A61K 8/21; A61P 31/04; A61K 31/722; A61Q 11/00

(52) U.S. Cl. 424/52; 424/49; 424/55

(57) ABSTRACT

An anti-bacterial oral care composition and method for producing the same are disclosed. The composition comprises chitosan, organic acid, bioflavonoids and xylitol that are mixed together and can effectively inhibit the growth of bacteria in oral cavity, such as 99.9% of staphylococcus aureus, 99.9% of streptococcus mutans, 99.9% of candida albicans, thus reducing the occurrence of decayed tooth. Furthermore, no chemical preservative and anti-bacterial agent are added in the composition, reducing the damage to the oral mucosa and the health of the user.

### BACKGROUND OF THE INVENTION

1. Field of the Invention

The present invention relates to an oral care composition and method for producing the same, and more particularly to an anti-bacterial oral care composition and method for producing the same.

2. Description of the Prior Art

Dental plaque is formed by polysaccharide generated from metabolism of bacteria in the oral cavity and bacteria enclosed therein. The dental plaque is difficult to clear due to its strong adherence and is the primary factor of causing the periodontal disease and decayed teeth. To inhibit the reproduction of dental plaque, the current oral health products normally contain anti-bacterial ingredient. In addition, killing bacteria and removing bad smell are two sides of a coin, in other words, if the reproduction of the bacteria is inhibited, it can prevent the bacteria from decomposing residue of food and producing the bad smell.

However, most of the current oral care products contain chemical preservative and anti-bacterial agent, taking the mouth rinse as an example, it kills bacteria, but it will also cause damage to the oral mucosa if used for too long at a time.

The present invention has arisen to mitigate and/or obviate the afore-described disadvantages.

### SUMMARY OF THE INVENTION

The primary objective of the present invention is to provide an anti-bacterial oral care composition and a method for producing the same, besides the anti-bacterial function, the anti-bacterial oral care composition can also reduce the damage to the damage to the oral mucosa and the health of the user.

Hence, to achieve the above objective, An anti-bacterial oral care composition comprising 0.5-30% wt. of chitosan, 0.5-30% wt. of organic acid, 0.1-25% wt. of bioflavonoids, 0.1-25% wt. of xylitol and 20-95% wt. of pure water.

Preferably, the weight ratio of the bioflavonoids is preferably 0.3-5%, and the weight ratio of the xylitol is preferably 1-5%.

Preferably, the organic acid is selected from the group consisting of formic acid, acetic acid, oxalic acid, malonic acid, succinic acid, tartaric acid, gluconic acid, malic acid, citric acid, lactic acid, and a combination thereof.

Preferably, acidulating chitosan formed after the chitosan is dissolved in the organic acid has a viscosity of 200 to 5000 cps at 10 rpm.

To achieve the above objective, a method for producing an anti-bacterial oral care composition comprising the steps of: mixing and dissolving: putting 0.5-30% wt. of chitosan and 0.5-30% wt. of organic acid in a sealing reaction tank, and then adding water in the reaction tank and stirring them; isolating oxidation: putting inert gas in the reaction tank for isolating air and avoiding oxidation; melting and forming: heating the reaction tank and controlling temperature of the reaction tank between 50° C. and 80° C. and reaction time between 60 and 120 minutes, continuously stirring the chitosan and the organic acid to form acidulating chitosan; and concocting: adding 0.1-25% wt. of bioflavonoids, 0.1-25% wt. of xylitol and 20-95% wt. of pure water in the acidulating chitosan and concocting them to form the liquid composition.

Preferably, the step of mixing and dissolving includes putting the chitosan in the sealing reaction tank and then adding water in the reaction tank and stirring them for 15-30 minutes, next putting the organic acid in the reaction tank and stirring them for 30-120 minutes for dissolving, the organic acid in the step of mixing and dissolving is selected from the group consisting of formic acid, acetic acid, oxalic acid, malonic acid, succinic acid, tartaric acid, gluconic acid, malic acid, citric acid, lactic acid, and a combination thereof.

Preferably, the inert gas in the step of isolating oxidation is selected from the group consisting of nitrogen, helium, neon, argon, krypton, xenon, and a combination thereof.

Preferably, the acidulating chitosan formed after the chitosan is dissolved in the organic acid has a viscosity of 200-5000 cps at 10 rpm.

Preferably, the step of concocting further includes adding 0.2-10% wt. of thickening agent to make the composition into paste, the thickening agent is selected from the group consisting of gelatine, agar, kanten, hydroxyethyl cellulose, hydroxypropyl cellulose, fydroxypropylmethyl cellulose, methyl cellulose, carboxymethyl cellulose, hydrophobically modified hydroxyethyl cellulose, and so on.

Preferably, the step of concocting further includes adding 20-60% wt. of abradant to achieve a function of oral decontamination and dental polish and make the composition into paste, and the abradant is selected from the group consisting of calcium carbonate, dicalcium phosphate, calcium pyrophosphate, and silica.

Preferably, the step of concocting further includes adding humectant, sodium chloride, sodium fluoride, surface active agent, sweetener, spice, and colorant.

### BRIEF DESCRIPTION OF THE DRAWINGS

FIG. 1 is a flow chart of a method for producing an anti-bacterial oral care composition in accordance with the present invention.

### DETAILED DESCRIPTION OF THE PREFERRED EMBODIMENTS

An anti-bacterial oral care composition in accordance with a preferred embodiment of the present invention comprises 0.5-30% wt. of chitosan, 0.5-30% wt. of organic acid, 0.1-25% wt. of bioflavonoids, 0.1-25% wt. of xylitol and 20-95% wt. of pure water. The weight ratio of bioflavonoids in accordance with the present invention is preferably 0.3-5%, and the weight ratio of the xylitol is preferably 1-5%. Acidulating chitosan formed after the chitosan is dissolved in the organic acid has a viscosity of 200 to 50000 cps at 10 rpm by acid degradation. The composition formed from the above components in accordance with the present invention is liquid.

The organic acid is selected from the group consisting of formic acid, acetic acid, oxalic acid, malonic acid, succinic acid, tartaric acid, gluconic acid, malic acid, citric acid, lactic acid, and a combination thereof.

Hereby, the composition of the present invention can effectively inhibit 99.9% of staphylococcus aureus, 99.9% of streptococcus mutans, and 99.9% of candida albicans, effectively inhibiting the growth of bacteria in oral cavity while reducing the occurrence of decayed teeth. Meanwhile, the present invention can prevent the bacteria from decomposing the food residue and generating source of foul smell by inhibiting the growth of bacteria, reducing oral malodor while maintaining fresh breath. Furthermore, no chemical preservative and anti-bacterial agent are added in the composition of the present invention, reducing damage to the oral mucosa and the health of the user.

The aforementioned is the main components and the function of the preferred embodiment in accordance with the present invention.

For a better understanding of the present invention, reference should be made to the following tests:

I. The anti-bacterial test result of the composition in accordance with the present invention against the staphylococcus aureus, streptococcus mutans and candida albicans is described as follows:

The anti-bacterial test is carried out according to U.S. Pharmacopeia 26 NF 21 Microbiological Tests(51)Antimicrobial Effectiveness Testing. The test result is shown in table 1, and the sample in the table 1 is the composition of the present invention. CFU/ml represents bacteria count per milliliter.

TABLE 1
Test result:
Test is carried out after sample is mixed with bacteria liquid in the
ratio of 1:1 and exposed for one hour

| | exposure time | | |
|---|---|---|---|
| | original | bacteria count | |
| | bacteria | after exposure | rate of inhibiting |
| bacteria strain | count | for one hour | bacteria (%) |
| Staphylococcus aureus | 2.9 × 10⁵ | not detected | >99.999 |
| ATCC6538 (CFU/ml) | | | |
| Streptococcus mutans | 3.8 × 10⁶ | not detected | >99.999 |
| ATCC31377 (CFU/ml) | | | |
| Candida albicans | 2.5 × 10⁶ | not detected | >99.999 |
| ATCC20512 (CFU/ml) | | | |

The above test was carried out in the following conditions:

1. preparing bacteria solution: taking 1 ml of suspension of the testing bacteria and adding it into 9 ml of axenic phosphate buffer and mixing them evenly, and then adding 1 ml to 100 ml of 0.5% Triton X-100, and finally mixing them evenly, bacteria concentration is 1×10⁵˜10⁶ organisms;

2. taking 1 m of sample and 1 ml of the above solution and injecting the sample and the bacteria solution at the ratio of 1:1 into a culture dish and mixing them evenly; and

3. waiting for 1 hour and then taking 1 ml of the sample into the culture dish, and injecting a proper amount of culture medium, finally mixing them evenly.

II. Test of viable count in the liquid composition in accordance with the present invention:

The test of viable count was carried out according to the method of test for food microbiology (CNS 10890 (2009 Jan. 17 revised edition), and the test result is shown in table 2. If the bacteria count is lower than the limit of detection, the test result will be represented by “negative” and “not detected”.

TABLE 2
Test result:

| | | | Detection | |
|---|---|---|---|---|
| Test item | Test method | Test result | limit | unit |
| Total bacteria count | CNS | Not | <1 | CFU/ml |
| | 10890(2009/01/17 | detected | | |
| | revised edition) | | | |

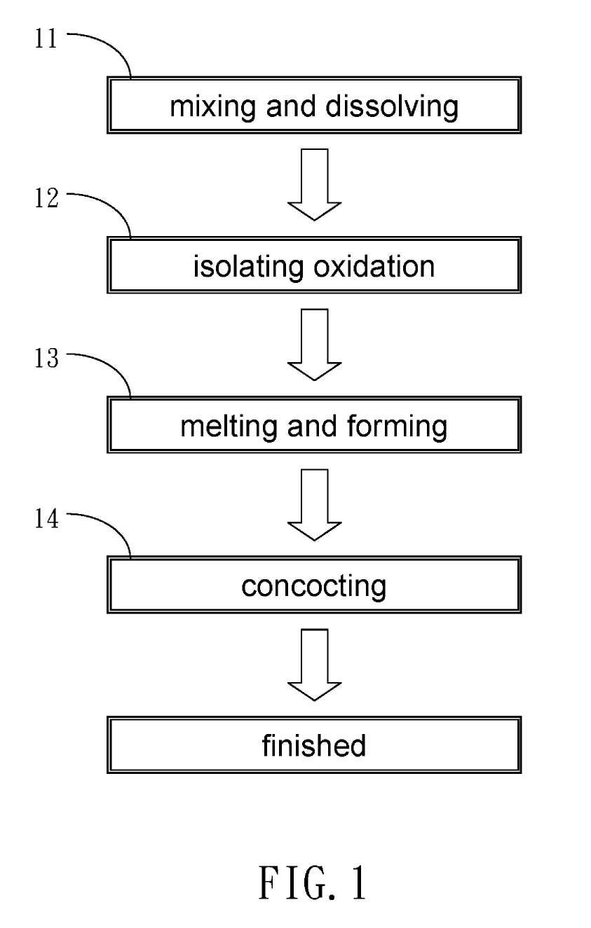

Referring to FIG. 1, a method for producing an anti-bacterial oral care composition in accordance with a preferred embodiment of the present invention comprises the steps of: mixing and dissolving 11, isolating oxidation 12, melting and forming 13, and concocting 14.

The step of mixing and dissolving 11: putting 0.5-30% wt. of chitosan in a sealing reaction tank and stirring it for 15-30 minutes, and then putting 0.5-30% wt. of organic acid in the reaction tank, and finally adding water in the reaction tank and stirring for 30-120 minutes.

The step of isolating oxidation 12: putting inert gas into the reaction tank for isolating the air and avoiding oxidation, the inert gas is selected from the group consisting of nitrogen, helium, neon, argon, krypton, xenon, and a combination thereof.

The step of melting and forming 13: heating the reaction tank and controlling the temperature of the reaction tank between 50° C. and 80° C. and the reaction time between 30 and 120 minutes, continuously stirring the chitosan and the organic acid to form acidulating chitosan having a viscosity of 200-20000 cps at 10 rpm by acid degradation, and the acidulating chitosan can be dissolved in water.

The step of concocting 14: adding 0.1-25% wt. of bioflavonoids, 0.1-25% wt. of xylitol and 20-95% wt. of pure water in the acidulating chitosan and concocting them to form the composition of the present invention, and the composition is the liquid. Preferably, the step of concocting further includes adding humectant, sodium chloride, sodium fluoride, surface active agent, sweetener, spice to concoct the liquid composition.

The aforementioned is the description of the method for producing an anti-bacterial oral care composition in accordance with the present invention.

It is to be noted that, in the above steps of the method in accordance with the present invention, the step of concocting 14 further includes adding 0.2-10% wt. of thickening agent to adjust the viscosity of the composition to form a paste composition, the thickening agent is selected from the group consisting of gelatine, agar, kanten, hydroxyethyl cellulose, hydroxypropyl cellulose, fydroxypropylmethyl cellulose, methyl cellulose, carboxymethyl cellulose, hydrophobically modified hydroxyethyl cellulose, and so on.

In the above steps, the step of concocting 14 further includes adding 20-60% wt. of abradant to achieve the function of oral decontamination and dental polish to make the composition in accordance with the present invention into paste, and the abradant may be calcium carbonate, dicalcium phosphate, calcium pyrophosphate, silica, and so on.

Preferably, the step of concocting 14 further includes adding humectant, sodium chloride, sodium fluoride, surface active agent, sweetener, spice, colorant to concoct the liquid composition.

The following description is directed to an anti-bacterial test of the paste composition in accordance with the present invention:

I. The anti-bacterial test result of the paste composition in accordance with the present invention against the staphylococcus aureus, streptococcus mutans and candida albicans is described as follows:

The anti-bacterial test is carried out according to AATC Test Method 30-2004 Antifungal Activity. The test result is shown in table 3, and CFU/ml represents bacteria count per milliliter.

TABLE 3
Test result:

| | Exposure time | | | |
|---|---|---|---|---|
| | | bacteria | | |
| | original | count after | | |
| | bacteria | culturing for | inhibition | inhibition |
| Bacteria strain name | count | 48 hours | zone | effect: |
| Staphylococcus aureus | 5.2 × 10⁷ | not detected | 10 mm | Excellent |
| ATCC6538 (CFU/ml) | | | | |
| Streptococcus mutans | 5.0 × 10⁷ | not detected | 12 mm | Excellent |
| ATCC31377 (CFU/ml) | | | | |
| Candida albicans | 1.5 × 10⁷ | not detected | 5 mm | Excellent |
| ATCC20512 (CFU/ml) | | | | |

The above test is carried out in the following conditions:

1. Spraying the paste composition in accordance with the present invention on a cloth surface, after being dry, cutting a test sample in a circle having a diameter of 38 mm, and finally culturing for 48 hours at 37° C. after inoculation.

2. The inhibition zone is calculated as follows:

W=(T−D)/2

W=width of inhibition zone mm

T=total width of test sample and inhibition zone mm

D=width of test sample mm

3.

| Test result | Growth of bacteria |
|---|---|
| Excellent | colony didn't grow within contact zone |
| Fair | a few colonies grew within contact zone |
| Unsatisfactory | colony multiplied within contact zone |

“˜” represents that the test result cannot be determined due to the tissue or color of the test sample surface.

II. The test of viable count for the paste composition in accordance with the present invention is carried out as follows:

The viable count test is carried out according to the method of test for food microbiology (CNS 10890 (2009 Jan. 17 revised edition), and the test result is shown in table 2. If the viable count is lower than the limit of detection, the test result will be represented by “negative” and “not detected”.

TABLE 4
Test result:

| | | | Detection | |
|---|---|---|---|---|
| Test item | Test method | Test result | limit | unit |
| Total viable count | CNS | Not | <1 | CFU/ml |
| | 10890(2009/01/17 | detected | | |
| | revised edition | | | |

To summarize, the composition in accordance with the present invention for dental cavity and gum care comprises plural natural materials, such as the chitosan which is extracted from converted from shrimp shell and crap shell and converted by a biochemical machine, and the bioflavonoids (commonly called vitamin P) which are extracted from the skin and the flesh of the natural citrus fruits. Both the two materials are natural anti-bacterial agents and can inhibit the harmful bacteria in the dental cavity at some extent. In addition, the xylitol which is the natural plant sweetener extracted from the silver birch, the oak and the like plant can effectively reduce the occurrence of decayed teeth and tartar. Since the glucose unit in the high polymer chain of the chitosan has amidocyanogen, the composition in accordance with the present invention can also effectively remove the acidic odor substance generated by the microorganisms decomposing the food residue.

While we have shown and described various embodiments in accordance with the present invention, it is clear to those skilled in the art that further embodiments may be made without departing from the scope of the present invention.

1. (canceled)

2. (canceled)

3. (canceled)

4. (canceled)

5. A method for producing an anti-bacterial oral care composition comprising the steps of:
mixing and dissolving: putting 0.5-30% wt. of chitosan and 0.5-30% wt. of organic acid in a sealing reaction tank, and then adding water in the reaction tank and stirring them;
isolating oxidation: putting inert gas in the reaction tank for isolating air and avoiding oxidation;
melting and forming: heating the reaction tank and controlling temperature of the reaction tank between 50° C. and 80° C. and reaction time between 60 and 120 minutes, continuously stirring the chitosan and the organic acid to form acidulating chitosan; and
concocting: adding 0.1-25% wt. of xylitol and 20-95% wt. of pure water in the acidulating chitosan and concocting them to form the liquid composition.

6. The method for producing an anti-bacterial oral care composition as claimed in claim 5, wherein the step of mixing and dissolving includes putting the chitosan in the sealing reaction tank and then adding water in the reaction tank and stirring them for 15-30 minutes, next putting the organic acid in the reaction tank and stirring them for 30-120 minutes for dissolving, the organic acid in the step of mixing and dissolving is selected from the group consisting of formic acid, acetic acid, oxalic acid, malonic acid, succinic acid, tartaric acid, gluconic acid, malic acid, citric acid, lactic acid, and a combination thereof.

7. The method for producing an anti-bacterial oral care composition as claimed in claim 5, wherein the inert gas in the step of isolating oxidation is selected from the group consisting of nitrogen, helium, neon, argon, krypton, xenon, and a combination thereof.

8. The method for producing an anti-bacterial oral care composition as claimed in claim 5, wherein the acidulating chitosan formed after the chitosan is dissolved in the organic acid has a viscosity of 200-5000 cps at 10 rpm.

9. The method for producing an anti-bacterial oral care composition as claimed in claim 5, wherein the step of concocting further includes adding 0.2-10% wt. of thickening agent to make the composition into paste, the thickening agent is selected from the group consisting of gelatine, agar, kanten, hydroxyethyl cellulose, hydroxypropyl cellulose, fydroxypropylmethyl cellulose, methyl cellulose, carboxymethyl cellulose, hydrophobically modified hydroxyethyl cellulose, and so on.

10. The method for producing an anti-bacterial oral care composition as claimed in claim 5, wherein the step of concocting further includes adding 20-60% wt. of abradant to achieve a function of oral decontamination and dental polish and make the composition into paste, and the abradant, is selected from the group consisting of calcium carbonate, dicalcium phosphate, calcium pyrophosphate, and silica.

11. The method for producing an anti-bacterial oral care composition as claimed in claim 5, wherein the step of concocting further includes adding humectant, sodium chloride, sodium fluoride, surface active agent, sweetener, spice, and colorant.

12. The method for producing an anti-bacterial oral care composition as claimed in claim 9, wherein the step of concocting further includes adding humectant, sodium chloride, sodium fluoride, surface active agent, sweetener, spice, and colorant.

13. The method for producing an anti-bacterial oral care composition as claimed in claim 10, wherein the step of concocting further includes adding humectant, sodium chloride, sodium fluoride, surface active agent, sweetener, spice, and colorant.

14. The method for producing an anti-bacterial oral care composition as claimed in claim 5, wherein the step of concocting further includes adding 0.1-25% wt. of bioflavonoids.
